(19) United States
(12) Patent Application Publication
(10) Pub. No.: US 2003/0118617 A1
(43) Pub. Date: Jun. 26, 2003

(54) Resveratrol analogues

(75) Inventors: Lynn Soby, South Nyack, NY (US); Eugenia H. Theophilus, Clemmons, NC (US); Dmitri Ptchelintsev, Jersey City, NJ (US); Paul S. Attar, Hawthorne, NJ (US)

Correspondence Address: Charles N.J. Ruggiero, Esq., Ohlandt, Greeley, Ruggiero & Perle, L.L.P., 10th Floor, One Landmark Square, Stamford, CT, 06901-2682, US

(73) Assignee: AVON PRODUCTS, INC., NEW YORK, NY (US)

(21) Appl. No.: 10/032,151

(22) Filed: Dec. 21, 2001

(51) Int. Cl. A61K 31/70; A61K 7/00; A61K 31/277; A61K 31/198; A61K 31/05; A61K 31/075

(52) U.S. Cl. 424/401; 514/25; 514/520; 514/568; 514/733; 514/720

(57) ABSTRACT

A method for treating the skin of a human is disclosed. Also disclosed is a composition for carrying out such method, in which the composition has at least one resveratrol analogue represented by the formula

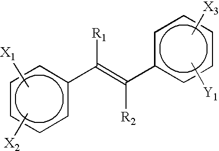

wherein each X₁ and X₂ are not both hydrogen but each X₁ and X₂ are otherwise independently selected from the group consisting of hydrogen, hydroxy, alkoxy, acyl and carboxyl; wherein each X₃ is selected from the group consisting of hydrogen, hydroxy, alkoxy and acyl; wherein each Y₁ is selected from the group consisting of carbohydrate, hydrogen and hydroxyl; wherein R₁ and R₂ are double-bonded in cis or trans configuration and each R₁ and R₂ are independently selected from the group consisting of hydrogen, alkoxy, carboxyl, methoxy, nitryl, isonitryl and cyano.

### BACKGROUND OF THE INVENTION

1. Field of the Invention

The present invention relates to improving the aesthetic appearance of skin. More particularly, the present invention relates to reducing, preventing, ameliorating, and/or inhibiting (hereinafter “treating”) the cosmetic signs of dermatological aging via topical application of at least one resveratrol analogue or compositions having same.

2. Description of the Prior Art

Human skin has two layers, namely, a superficial or upper layer called the epidermis, and a deep layer called the dermis.

The dermis provides a solid support for the epidermis. It is also the feeder layer for the epidermis. The dermis mainly has fibroblasts and an extracellular matrix. The matrix is made of various extracellular proteins. Such proteins include, in particular, collagen fibers, elastin fibers and various glycoproteins. All of these extracellular species are synthesized by the fibroblast. Also present in the dermis are leukocytes, mastocytes or tissue macrophages. Finally, the dermis also has blood vessels and nerve fibers. The fibroblast, by virtue of its activity in the synthesis of extracellular matrix proteins (proteoglycans, collagen fibers and other structural glycoproteins), is the primary cellular constituent in the structural assembly of the dermis. As skin ages, whether intrinsically or extrinsically, such as by photoaging, cellular aging is taking place.

Moreover, at menopause, the principal modifications regarding the dermis are a reduction in the collagen level and in the dermal thickness. This causes, in menopausal women, a reduction in the thickness of the skin. Women then experience a sensation of “dry skin” or of tight skin and a marked increase in surface fine lines and fine wrinkles is observed. The skin exhibits a rough appearance upon palpation. Also, the skin exhibits reduced suppleness.

In the prior art, resveratrol analogues are used, inter alia, as depigmenting agents (JP-87/192040), as vasodilating agents (EP-96/830517), as antithrombotic agents (JP-05/016413), in the treatment of various cardiovascular conditions (CA 2187990), as mutagenesis and carcinogenesis inhibiting agents (JP-06/024967), or, alternatively, are described as antioxidants. Furthermore, hydroxystilbenes are utilized to stimulate the synthesis of collagen and/or the proliferation of the fibroblasts of the dermis and/or the inhibition of the expression of proteases of the extracellular matrix (U.S. Pat. No. 6,147,121).

Among these compounds, resveratrol (or 3,4′,5-trihydroxystilbene) is of particular interest for the activities described above mainly because it is a natural compound. The review by Soleas et al., Clinical Biochemistry, Vol. 30, No. 2, pp. 91-113 (1997) perfectly summarizes the state of the art respecting this compound and the resveratrol analogues generically.

### SUMMARY OF THE INVENTION

It is an object of the present invention to provide the use of an effective amount of at least one resveratrol analogue or composition having same to treat the dermatological signs of aging.

It is yet another object of the present invention to treat the appearance of sagging and/or wrinkled skin by topically applying to the skin at least one resveratrol analogue or composition having same to treat such sagging and/or wrinkled skin.

It is still another object of the present invention to provide the use of an effective amount of at least one resveratrol analogue or composition having same to improve the aesthetic appearance of skin.

It is a further object of the present invention to provide the use of an effective amount of at least one resveratrol analogue or composition having same to promote the smoothing of the skin and/or to firm the skin.

It is a further object of the present invention to provide the use of an effective amount of at least one resveratrol analogue or composition having same to combat the dermatological effects of aging, more particularly the effects of menopause on collagen and/or fibroblasts.

These and other objects and advantages of the present invention are achieved by a composition having one or more resveratrol analogues that is a compound having the following structural formula (I):

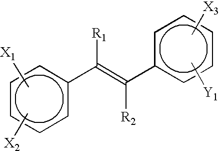

in which each X₁ and X₂ are not both hydrogen but each X₁ and X₂ are otherwise independently selected from the group consisting of hydrogen, hydroxy, alkoxy, acyl and carboxyl. Each X₃ is selected from the-group consisting of hydrogen, hydroxy, alkoxy and acyl. Each Y₁ is selected from the group consisting of carbohydrate, hydrogen arid hydroxyl. Each R₁ and R₂ is independently selected from the group consisting of hydrogen, alkoxy, carboxyl, methoxy, nitryl, isonitryl and cyano. These compounds may be in a cis- or trans-configuration.

### DETAILED DESCRIPTION OF THE INVENTION

The present invention provides anti-aging benefits to and improves the aesthetic appearance of skin. In particular, the present invention provides compositions and methods for treating skin to treat the signs of dermatological aging due to, for example, chronological aging, hormonal aging, and/or photoaging. Such signs of aging include, but are not limited to, skin fragility; loss of collagen and/or elastin; estrogen imbalance in skin; skin atrophy; appearance and/or depth of lines and/or wrinkles, including fine lines; skin discoloration, including dark eye circles; skin sagging; skin fatigue and/or stress, e.g. skin breakout due to environmental stress, such as pollution and/or temperature changes; skin dryness; skin flakiness; cellular aging; loss of skin tone, elasticity and/or luster; loss of skin firmness; poor skin texture; loss of skin elasticity and/or resiliency; and thin skin.

The benefits and improvements to the aesthetic appearance of skin can be manifested in any of the following: reduction in pore size; improvement in skin tone, radiance, clarity and/or tautness; promotion of anti-oxidant activity; improvement in skin firmness, plumpness, suppleness, and/or softness; improvement in procollagen and/or collagen production; improvement in skin texture and/or promotion of retexturization; improvement in skin barrier repair and/or function; improvement in appearance of skin contours; restoration of skin luster and/or brightness; replenishment of essential nutrients and/or constituents in the skin decreased by aging and/or menopause; improvement in communication among skin cells; increase in cell and/or fibroblast proliferation and/or multiplication; increase in skin cell metabolism decreased by aging and/or menopause; improvement in skin moisturization; promotion and/or acceleration of cell turnover; enhancement of skin thickness; increase in skin elasticity and/or resiliency; and enhancement of exfoliation, with or without the use of alpha or beta hydroxy acids, keto acids or other exfoliants.

The present invention relates to treating skin with the topical application of one or more resveratrol analogues, preferably in a composition having a cosmetically acceptable vehicle.

Among the particularly preferred resveratrol analogues of structural formula (I) include compounds in which X₁ and X₂ are preferably independently selected from hydroxy and alkoxyl groups, with hydroxy, methoxy, ethoxy, propoxy, isopropoxy, butoxy and isobutoxy groups being preferred. Most preferred, X₁ and X₂ are hydroxy and methoxy groups. Each X₃ is preferably hydroxy, methoxy, propoxy, isopropoxy, butoxy and isobutoxy groups, with hydroxy and methoxy groups being most preferred. Y₁ is preferably a carbohydrate. R₁ and R₂ are preferably independently hydrogen and cyano, and hydrogen is most preferred. R₁ and R₂ may not both be hydrogen. In yet another preferred structure, X₁=OH, X₂=OCH₃, X₃=OH and Y₁=a carbohydrate. Further, X₁ and X₂ are ortho to one another, and X₃ and Y₁ are meta to one another. Another preferred structure of the present invention has X₁=H, X₂=OH, Yi=H and X₃=OCH₃ with X₂ in a para position and X₃ in a meta position. In still another preferred embodiment, X₁=OH, R₁=CN, and X₃=X₁=OCH₃. In all cases, these compounds are preferably in the trans-configuration.

According to the present invention, the resveratrol analogues may be used either alone or in the form of mixtures of any type, and may be natural or synthetic in origin.

The resveratrol analogues may be formulated into cosmetic and/or pharmaceutical, particularly dermatological, compositions suited to treat aging skin, particularly to stimulate the synthesis of collagen and/or the proliferation of the fibroblasts of the dermis.

The amount of resveratrol analogue administered according to the present invention depends on the desired effect. However, the amount should be effective to bring about the intended result. Also, the resveratrol analogue and/or the composition containing the analogue, should be topically applied for a period of time sufficient to bring about the intended result.

For example, with respect to the amount of resveratrol analogue administered according to the present invention, it advantageously ranges about 0.00001 percentage by weight (wt %) to about 50 wt % based on the total weight of the composition. Preferably, the amount of resveratrol analogue is about 0.0001 wt % to about 20 wt % of the total weight of the composition. Most preferably, the amount of resveratrol analogue is about 0.001 wt % to about 10 wt % of the total weight of the composition. With respect to the amount of time such a composition would be topically applied to skin, it advantageously includes at least daily application for a period of 1, 2 or 4 weeks.

The compositions of the present invention may have a cosmetically acceptable medium. Cosmetically acceptable medium means a vehicle, diluent or carrier, which is compatible with the skin, the nails and/or the hair.

The compositions according to the present invention may be formulated in all dosage forms normally employed for topical application. Among the forms embodied by the present invention are the following: aqueous, aqueous/alcoholic, or oily solutions; oil-in-water, water-in-oil, water-in-silicone, silicone-in-water, or multiple emulsions; aqueous or oily gels; anhydrous liquids; pasty or solid products; and dispersions of oil in an aqueous phase with the aid of spherules. The spherules of the present invention may be polymeric nanoparticles, such as nanospheres and nanocapsules, or, more preferably, lipid vesicles of the ionic and/or nonionic type.

The present compositions may be fluid to a greater or lesser degree. For example, the present compositions may have the form of a cream, an emulsion, a foam, a gel, a lotion, a milk, a mousse, an ointment, a solution, a paste, a pomade, a powder, a serum, or incorporated into a patch or towelette. Such compositions may optionally be applied to the skin in the form of an aerosol or spray pump.

They may also be provided in solid form, for example in the form of a stick. Regardless of the solidity of compositions of the present invention, these compositions may be used as a treatment, a cleansing, or a makeup product.

Other actives may be used in compositions having resveratrol analogues of the present invention. Such active agents include, but are not limited to, one or more agents modulating bacterial adhesion to the skin and/or mucous membranes such as honey, especially honey derived from acacias and certain sugar derivatives; keratolytic agents such as alpha- and beta-hydroxycarboxylic or beta-ketocarboxylic acids, their salts, amides or esters and more particularly hydroxy acids such as gylcolic acid, lactic acid, salicyclic acid, citric acid and, in general, fruit acids and 5-n-octanoylsalicylic acid; anti-free radical agents, such as alphatocopherol and its esters, superoxide dismutases, certain metal chelators or ascorbic acid and its esters; anti-acne agents such as benzoyl peroxide; substances such as Substance P or bradykinin antagonists or NO synthase inhibitors or, alternatively, sodium channel inhibitors, compounds described as being active in the treatment of sensitive skins and as having anti-irritant effects.

Optionally, compositions useful in the present method can include one or more of the following additional ingredients: amino acids, anesthetics, anti-allergenics, antifungals, antimicrobials, anti-inflammatory agents, antineoplastics, antioxidants, antiseptics, antivirals, chelating agents, colorants, depigmenting agents, emollients, emulsifiers, film formers, fragrances, humectants, hypopigmenting agents, immune system boosting agents, immune system suppressing agents, insect repellents, lubricants, matting agents, moisturizers, pharmaceutical agents, photostabilizing agents, preservatives, retinoids, skin protectants, skin penetration enhancers, staining agents, sunscreens, stabilizers, surfactants, thickeners, viscosity and/or rheology modifiers, vitamins, or any combinations thereof.

As the skin contains many components, in addition to collagen and fibroblasts, it is advantageous, when a resveratrol analogue according to the present invention is topically applied, to promote at the same time the synthesis of these other components, such as lipids and/or the proliferation of other cellular components such as keratinocytes. In addition to improving the aesthetic appearance of skin, and treating the signs of aging skin, the present invention intends particularly to bring about improvements in elastase inhibition, collagenase inhibition and fibroblast proliferation by improving collagen production, lipid peroxidation and metabolism boost.

While the present invention has been described in terms of various preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A composition for treating skin, said composition comprising at least one resveratrol analogue represented by the formula 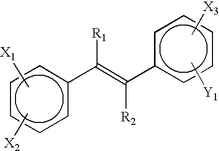
wherein each X₁ and X₂ are not both hydrogen but each X₁ and X₂ are otherwise independently selected from the group consisting of hydrogen, hydroxy, alkoxy, acyl and carboxyl;
wherein each X₃ is selected from the group consisting of hydrogen, hydroxy, alkoxy and acyl;
wherein each Y₁ is selected from the group consisting of carbohydrate, hydrogen and hydroxyl;
wherein R₁ and R₂ are double-bonded in cis or trans configuration and each R₁ and R₂ are independently selected from the group consisting of hydrogen, alkoxy, carboxyl, methoxy, nitryl, isonitryl and cyano; and
a cosmetically acceptable vehicle suitable for topical application to skin.

2. The composition of claim 1, wherein said resveratrol analogue is present in an amount about 0.00001 wt % to about 50 wt % of the total weight of the composition.

3. The composition of claim 1, wherein said resveratrol analogue is present in an amount about 0.0001 wt % to about 20 wt % of the total weight of the composition.

4. The composition of claim 1, wherein said resveratrol analogue is present in an amount about 0.001 wt % to about 10 wt % of the total weight of the composition.

5. The composition of claim 1, wherein said x₁ and x₂ are selected from the group consisting of hydroxy, methoxy, ethoxy, propoxy, isopropoxy, butoxy and isobutoxy.

6. The composition of claim 5, wherein said x₁ and x₂ are hydroxy and methoxy.

7. The composition of claim 1, wherein said x₃ is selected from the group consisting of hydroxy, methoxy, propoxy, isopropoxy, butoxy and isobutoxy.

8. The composition of claim 7, wherein said X₃ is hydroxy or methoxy.

9. The composition of claim 1, wherein Y1 is a carbohydrate.

10. The composition of claim 1, wherein R₁ and R₂ are selected from the group consisting of hydrogen and cyano.

11. The composition of claim 10, wherein R₁ or R₂ is hydrogen.

12. The composition of claim 1, wherein X₁ is hydroxy, X₂ is methoxy, X₃ is hydroxy and Y₁ is a carbohydrate.

13. The composition of claim 12, wherein X₁ and X₂ are ortho to one another, and X₃ and Y₁ are meta to one another.

14. The composition of claim 1, wherein X₁ is hydrogen, X₂ is hydroxy, Y₁ is hydrogen and X₃ is methoxy.

15. The composition of claim 14, wherein X₂ is in a para position and X₃ is in a meta position.

16. The composition of claim 1, wherein X₁ is hydroxy, R₁ is cyano, X₃ is methoxy and X₁ is methoxy.

17. The composition of claim 1, wherein said resveratrol analogue is in the trans configuration.

18. The composition of claim 1, further comprising a sunscreen agent.

19. A method of improving the aesthetic appearance of skin, comprising topically applying to said skin the composition of claim 1 in an amount effective and for a period of time sufficient to bring about such improvement.

20. The method of claim 19, wherein the composition is applied at least once daily.

21. The method of claim 19, wherein the composition is applied daily for about two weeks.

22. The method of claim 19, wherein the composition is applied daily for about 4 weeks.

23. The method of claim 19, wherein said resveratrol analogue is present in an amount about 0.00001 wt % to about 50 wt % of the total weight of the composition.

24. The method of claim 19, wherein said resveratrol analogue is present in an amount about 0.0001 wt % to about 20 wt % of the total weight of the composition.

25. The method of claim 19, wherein said resveratrol analogue is present in an amount about 0.001 wt % to about 10 wt % of the total weight of the composition.

26. The method of claim 19, wherein the improvements are one or more improvements selected from the group consisting of reduction in dermatological signs of chronological aging, hormonal aging and/or photo aging; prevention and/or reduction in appearance and/or depth of lines and/or wrinkles; improvement in skin tone, radiance, clarity and/or tautness; improvement in skin firmness, plumpness, suppleness, and/or softness; and improvement in skin texture and/or promotion of retexturization.

27. The method of claim 19, wherein the improvements are one or more improvements selected from the group consisting of minimization of dermatological signs of fatigue and/or stress; resistance to environmental stress; improvement in procollagen and/or collagen production; prevention and/or reversal of loss of collagen and/or elastin; minimization of skin dryness and/or improvement in skin moisturization; enhancement of skin thickness; increase in skin elasticity and/or resiliency; ameliorating the effects of estrogen imbalance in the skin; and enhancement of exfoliation.

28. The method of claim 19, wherein the improvements are one or more improvements selected from the group consisting of reduction in dermatological signs of chronological aging, hormonal aging and/or photoaging; reduction in skin fragility; reduction in pore size; prevention and/or reversal of loss of collagen and/or elastin; ameliorating the effects of estrogen imbalance on skin; prevention of skin atrophy; prevention and/or reduction in appearance and/or depth of lines and/or wrinkles; prevention, reduction and/or treatment of hyperpigmentation; improvement in skin tone, radiance, clarity and/or tautness; prevention, reduction, and/or amelioration of skin sagging; promotion of anti-oxidant activity; improvement in skin firmness, plumpness, suppleness and/or softness; improvement in procollagen and/or collagen production; improvement in skin texture and/or promotion of retexturization; improvement in skin barrier repair and/or function; improvement in appearance of skin contours; restoration of skin luster and/or brightness; minimization of dermatological signs of fatigue and/or stress; resistance to environmental stress; replenishment of essential nutrient and/or constituents of in the skin decreased by aging and/or menopause; improvement in communication among skin cells; increase in cell proliferation and/or multiplication; increase in skin cell metabolism decreased by aging and/or menopause; retardation of cellular aging; inhibition of enzymes in the skin that accelerate aging of skin cells; minimization of skin dryness and/or improvement in skin moisturization; minimization of skin discoloration; promotion and/or acceleration of cell turnover; enhancement of skin thickness; increase in skin elasticity and/or resiliency; and enhancement of exfoliation.

29. The method of claim 28, wherein said resveratrol analogue is present in an amount about 0.00001 wt % to about 50 wt % of the total weight of the composition.

30. The method of claim 28, wherein said resveratrol analogue is present in an amount about 0.0001 wt % to about 20 wt % of the total weight of the composition.

31. The method of claim 28, wherein said resveratrol analogue is present in an amount about 0.001 wt % to about 10 wt % of the total weight of the composition.

32. A method of treating the dermatological signs of aging skin comprising topically applying to the skin an effective amount of the composition of claim 1.

33. The method of claim 32, wherein the signs of aging skin are selected from the group consisting of skin fragility; loss of collagen and/or elastin; estrogen imbalance in skin; skin atrophy; appearance and/or depth of lines and wrinkles; skin discoloration; skin sagging; skin fatigue and/or stress; skin dryness; skin flakiness; cellular aging; loss of skin tone, clarity and/or luster; loss of skin firmness; poor skin texture; loss of skin elasticity and/or resiliency; and thin skin.

34. The method of claim 32, wherein said resveratrol analogue is present in an amount about 0.00001 wt % to about 50 wt % of the total weight of the composition.

35. The method of claim 32, wherein said resveratrol analogue is present in an amount about 0.0001 wt % to about 20 wt % of the total weight of the composition.

36. The method of claim 32, wherein said resveratrol analogue is present in an amount about 0.001 wt % to about 10 wt % of the total weight of the composition.

37. The method of claim 32, wherein the signs of aging are the result of menopause.

38. The method of claim 32, wherein the signs of aging skin are ameliorated by an improvement selected from the group consisting of elastase inhibition, collagenase inhibition and fibroblast proliferation.
